(19) United States
(12) Patent Application Publication
(10) Pub. No.: US 2022/0062218 A1
(43) Pub. Date: Mar. 3, 2022

(54) COMPOSITIONS AND METHODS FOR TREATING DIABETES

(71) Applicant: ENZYCHEM LIFESCIENCES, Seoul (KR)

(72) Inventors: Ki Young Sohn, Seoul (KR); Jae Wha Kim, Daejeon (KR); Sun Young Yoon, Daejeon (KR)

(21) Appl. No.: 17/423,218

(22) PCT Filed: Apr. 16, 2020
(86) PCT No.: PCT/IB20/53627

§ 371 Date: Jul. 15, 2021

Related U.S. Application Data
(60) Provisional application No. 62/908,382, filed on Sep. 30, 2019.

(30) Foreign Application Priority Data
Apr. 16, 2019 (KR) 10-2019-0044514

(51) Int. Cl. A61K 31/231; A61K 45/06; A61P 3/10

(52) U.S. Cl. CPC A61K 31/231; A23L 33/12; A61P 3/10; A61K 45/06

(57) ABSTRACT

Disclosed are methods and compositions for treating diabetes. The composition comprises a monoacetyldiacylglycerol compound of Formula 1 as an active ingredient for treating diabetes. [Formula 1] wherein R1 and R2 are independently a fatty acid residue of 14 to 22 carbon atoms.

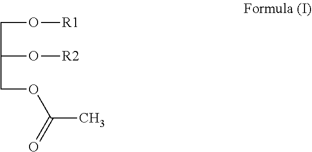

### CROSS-REFERENCE TO RELATED APPLICATION

This application claims benefit of priority to U.S. Provisional Application No. 62/908,382 filed on Sep. 30, 2019, and the benefit of priority to Korean Patent Application No. 10-2019-0044514 filed on Apr. 16, 2019, the entire contents of which applications are incorporated herein by reference.

### FIELD

This invention relates to a composition comprising a monoacetyldiacylglycerol compound for treating diabetes and more particularly to a composition comprising monoacetyldiacylglycerol compound for oral administration that can treat diabetes and alleviate the symptoms of diabetes.

### BACKGROUND

Diabetes mellitus is a chronic disease resulting from abnormalities in glucose, lipid and/or amino acid metabolism due to failure of insulin function. Diabetes is largely classified into type I diabetes (insulin dependent diabetes mellitus: IDDM) in which β cells on the Langerhans' island are destroyed and insulin secretion is irreversibly reduced to become hyperglycemia, and type II diabetes (non-insulin dependent diabetes mellitus: NIDDM) in which the insulin response to glucose decreases or insulin resistance to glucose increases in the Langerhans' island, and thereby resulting in chronic hyperglycemia.

Glucose is one of body's main energy sources and is used by most cells and plays an important role in cell function. When the glucose is ingested, blood glucose levels increase to secrete insulin in pancreatic beta cells. In response to the secreted insulin, the blood glucose is absorbed into muscle or adipose tissue so as to be used as an energy source. However, the increased blood glucose levels have a detrimental effect on function and survival of pancreatic beta cells. The high concentration of glucose induces overstimulation of beta cells, which makes the insulin synthesis rate slower than the insulin secretion rate. As a result, glucose-stimulated insulin secretion (GSIS), the most important function of beta cells, does not work properly. In addition, the high glucose concentration does not only induce oxidative stress, endoplasmic reticulum stress, or apoptosis, but also inhibits the cell differentiation, thus adversely affecting the survival of beta cells.

It would be desirable to have new therapies for diabetes.

### SUMMARY

In one aspect, compositions and methods are provided for treating diabetes comprising a monoacetyldiacylglycerol compound. In preferred methods and compositions, damage of beta cells due to excessive glucose uptake can be alleviated or mitigated by promoting the endocytosis of glucose transporter 2 (GLUT2) in pancreatic beta cells.

In another aspects, compositions and methods are for treating diabetes comprising a monoacetyldiacylglycerol compound that is not toxic and alleviates the symptoms of diabetes.

More particularly compositions and methods are provided comprising a monoacetyldiacylglycerol compound of Formula 1 for treating diabetes.

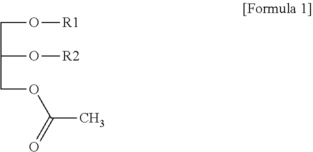

wherein R₁ and R₂ are independently a fatty acid residue of 14 to 22 carbon atoms, preferably a fatty acid residue of 15 to 20 carbon atoms.

In one embodiment, the monoacetyldiacylglycerol is a compound of the following Formula 2:

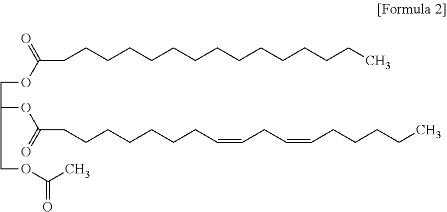 [SMILES: CCCCC/C=C\C/C=C\CCCCCCCC(=O)OC(COC(C)=O)COC(=O)CCCCCCCCCCCCCCC]

The compound of Formula 2 is 1-palmitoyl-2-linoleoyl-3-acetyl-rac-glycerol and corresponds to the compound of Formula 1 in which R₁ and R₂ of Formula 1 are palmitoyl and linoleoyl, respectively. The compound of Formula 2 is sometimes referred as “PLAG” or “EC-18” in this disclosure.

The present invention also provides a health functional food composition comprising a monoacetyldiacylglycerol compound of the Formula 1 or Formula 2 for alleviating or preventing diabetes and a method of treating diabetes comprising administering the composition to a suspected subject of diabetes disease.

In certain preferred aspects, a composition for treating diabetes containing the monoacetyldiacylglycerol compound (i.e. a compound of Formulae 1 or 2) according to the present invention promotes the endocytosis of glucose transporter 2 (GLUT2), and thereby attenuates beta cell damage of the pancreas due to the excessive glucose uptake.

As discussed, compounds of Formulae I and 2 may be used to treat a subject suffering from or susceptible to diabetes, including type I or type lI diabetes.

In a particular aspect, a compound of Formula I or 2 is used to treat a pre-diabetic patient.

In a further aspect, a compound of Formula I or 2 is used to treat a subject suffering from or susceptible to type 2 diabetes mellitus; diabetic dislipidemia; impaired or inadequate glucose tolerance (IGT); impaired fasting plasma glucose (IFG); metabolic acidosis; ketosis; appetite regulation; obesity; complications associated with diabetes including diabetic neuropathy, diabetic retinopathy and kidney disease; hyperlipidemia including hypertriglyceridemia, hypercholesteremia, and postprandial hyperlipidemia; arteriosclerosis; and hypertension,

In a yet further aspect, a compound of Formula 1 or 2 is used to treat a subject suffering from or susceptible to insulin resistance, hyperglycemia, hyperlipidemia, hypercholesterolemia, dyslipideinia, syndrome X or metabolic syndrome.

In particular aspects, a subject will be identified and selected for treatment of a disease or disorder as disclosed herein, and then a compound of Formula 1 or 2 will be administered to the identified and selected subject. For instance, a patient may be identified and selected as suffering from type 2 diabetes and that patient identified as suffering from type 2 diabetes may be administered a compound of Formula 1 or 2 to thereby alleviate or teat the type 2 diabetes.

In certain aspects, the present therapeutic methods are not associated with treatment of a subject suffering from a wound or injured tissue. In this aspect, subjects that are suffering from a wound or injured tissue and/or are seeking treatment for a wound or injured tissue would be excluded from the present therapeutic methods. In a related aspect, subjects seeking treatment involving tissue repair or regeneration would be excluded from the present therapeutic methods.

In a further aspect, pharmaceutical compositions are provided comprising a compound of Formula 1 or 2 as set forth above. The compositions suitably may comprise one or more pharmaceutically acceptable carriers. In preferred embodiments, the compositions may be formulated or otherwise adapted for treatment of diabetes or other disease or disorder as disclosed herein. In preferred aspects, the composition may be adapted for oral administration as a tablet or capsule.

In a yet further aspect, kits are provided for use to treat or prevent diabetes or other disease or disorder as disclosed herein. Kits of the invention suitably may comprise 1) one or more compounds of Formulae 1 or 2; and 2) instructions for using the one or more compounds for treating or preventing diabetes or other disorder or disease as disclosed herein. Preferably, a kit will comprise a therapeutically effective amount of one or more compounds of Formulae 1 or 2. The instructions suitably may be in written form, including as a product label.

In another aspect, compositions for combination therapy are provided. The composition includes:

i) a compound of Formula 1 for treating diabetes;

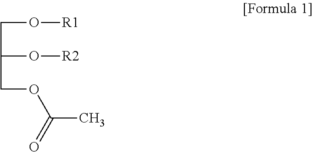

wherein R1 and R2 are independently a fatty acid residue of 14 to 22 carbon atoms; and

ii) one or more diabetic medications or diabetic treating agents.

In further aspect, methods for treating a subject suffering from or susceptible to diabetes are provided. The method includes administering to the subject an effective amount of a compound of Formula 1:

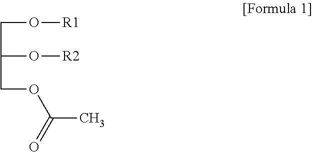

wherein R1 and R2 are independently a fatty acid residue of 14 to 22 carbon atoms; and

administering to the subject an effective amount of one or more diabetic medications or diabetic treating agents.

In another aspect, provided are methods of treating a subject suffering from or susceptible to type 2 diabetes mellitus; diabetic dyslipidemia; impaired or inadequate glucose tolerance (IGT); impaired fasting plasma glucose (IFG); metabolic acidosis; ketosis; obesity; diabetic neuropathy; diabetic retinopathy and kidney disease; hyperlipidemia; arteriosclerosis; hypertension; insulin resistance; hyperglycemia; hypercholesterolemia; dyslipidemia, or syndrome X.

The methods include administering to the subject an effective amount of a compound of Formula 1:

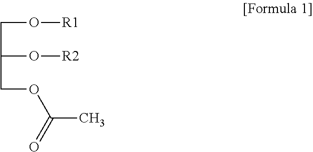

wherein R1 and R2 are independently a fatty acid residue of 14 to 22 carbon atoms; and
administering to the subject an effective amount of one or more diabetic medications or diabetic treating agents.

In certain aspects, one or more compounds of Formula 1 or 2, or PLAG may be administered to a subject in combination or coordination with one or more diabetes treatment agents that are distinct from the one or more compounds of Formula 1 or 2, or PLAG.

In another aspect, kits including (a) PLAG; (b) one or more diabetic medications or diabetic treating agents; and (c) instructions for using the PLAG for treating or preventing diabetes are provided.

Other aspects of the invention are disclosed infra.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 (includes FIGS. 1A through 1D) shows a chart of blood glucose levels (FIG. 1A), a chart of serum insulin (FIG. 1B), a chart of body weight changes measured (FIG. 1C), and images of pancreatic tissue stained (FIG. 1D), when a composition according to an embodiment of the present invention is administered.

FIG. 2 (includes FIGS. 2A through 2C) shows charts showing cell apoptosis in INS-1 cells, analyzed by flow cytometry (FIG. 2A), charts of cell apoptosis (FIG. 2B), and a chart of expression of apoptosis-related proteins like BAX, cytochrome c and caspase-3 (FIG. 2C), when a composition according to an embodiment of the present invention is administered.

FIG. 3 (includes FIGS. 3A through 3C) shows charts of expression of Glucose Transporter 2 (GLUT2) and Rac 1 measured by western blotting (FIGS. 3A and 3B) and images of expression of Glucose Transporter 2 (GLUT2) observed with Immunofluorescence assay (FIG. 3C), when a composition according to an embodiment of the present invention is administered.

FIG. 4 (includes FIGS. 4A through 4E) shows charts of expression of reactive oxygen species (ROS) (FIG. 4A and FIG. 4B), images of ROS expression observed with Immunofluorescence assay (FIG. 4C) and a chart analyzing the relationship between production of Intracellular ROS and apoptosis in pancreatic beta cells (FIGS. 4D and 4E), when a composition according to an embodiment of the present invention is administered.

FIG. 5 (includes FIGS. 5A through 5C) shows charts of glucose uptake (FIG. 5A and FIG. 5B) and images of glucose uptake observed with Immunofluorescence assay (FIG. 5C), when a composition according to an embodiment of the present invention is administered.

FIG. 6 (includes FIGS. 6A through 6E) shows charts analyzed by flow cytometry for the association of GLUT2 expression with apoptosis, ROS production and glucose uptake, when a composition according to an embodiment of the present invention is administered.

FIG. 7 (includes FIGS. 7A through 7F) shows structural formula of PLAG according to the present invention and PLH (A) and charts showing the specificity of PLAG activity.

FIG. 8 shows that PLAG reduces high glucose-induced cell apoptosis. Cell apoptosis in INS-1 cells was analyzed by flow cytometry using annexin-V and 7-AAD dyes. The protective effect of PLAG on glucotoxicity-induced pancreatic beta cell damage was further investigated after high glucose (HG) treatment. Cell apoptosis was increased up to 35% in HG-treated cells, and PLAG dose-dependently decreased the HG-induced cell apoptosis.

FIG. 9 shows that PLAG reduces high glucose (HG)-induced intracellular ROS generation. Intracellular ROS generation in INS-1 cells was analyzed by flow cytometry using DCFH-DA dye. ROS generation also was increased in HG-treated cells, and dose-dependently decreased by PLAG treatment.

FIG. 10 shows that PLAG accelerated GLUT2 endocytosis in high glucose-treated INS-1 cells. Effect of PLAG on plasma membrane GLUT2 expression in high glucose-treated INS-1 cells. GLUT2 expression in membrane fractions was analyzed by Western blotting. HG stimulates ChREBP induction that activates the expression of GLUT2 and TXNIP (a-arrestin). In PLAG-treated cells, GLUT2 expression in plasma membrane gradually decreased until 15min and then recovered and returned to control levels at 60 min

### DETAILED DESCRIPTION

The composition for treating diabetes of the present invention comprises a monoacetyldiacylglycerol compound of Formula 1 as an active ingredient.

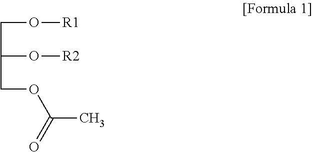

In the present invention, the term “monoacetyldiacylglycerol compound” means a glycerol derivative containing an acetyl group and two acyl groups and is also referred to as monoacetyldiacylglycerol (MADG).

In Formula 1, R₁ and R₂ are independently a fatty acid residue of 14 to 22 carbon atoms, preferably a fatty acid residue of 15 to 20 carbon atoms. The fatty acid residue means the remaining portion of the fatty acid in which the —OH group is excluded from its carboxyl group. In the Formula 1, non-limiting examples of R1 and R2 include palmitoyl, oleoyl, linoleoyl, linolenoyl, stearoyl, myristoyl, arachidonoyl, and so on. Preferable combinations of R1 and R2 include oleoyl/palmitoyl, palmitoyl/oleoyl, palmitoyl/linoleoyl, palmitoyl/linolenoyl, palmitoyl/arachidonoyl, palmitoyl/stearoyl, palmitoyl/palmitoyl, oleoyl/stearoyl, linoleoyl/palmitoyl, linoleoyl/stearoyl, stearoyl/linoleoyl, stearoyl/oleoyl, myristoyl/linoleoyl, myristoyl/oleoyl, and so on. More preferable combination of R1 and R2 is palmitoyl/linoleoyl. In optical activity, the monoacetyldiacylglycerol derivatives of Formula 1 can be (R)-form, (S)-form or a racemic mixture, preferably a racemic mixture, and may include their stereoisomers.

In one embodiment, the monoacetyldiacylglycerol is a compound of the following Formula 2:

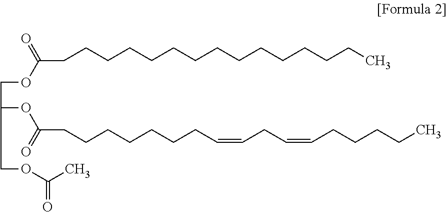 [SMILES: CCCCC/C=C\C/C=C\CCCCCCCC(=O)OC(COC(C)=O)COC(=O)CCCCCCCCCCCCCCC]

The compound of Formula 2 is 1-palmitoyl-2-linoleoyl-3-acetyl-rac-glycerol and corresponds to the compound of Formula 1 in which R₁ and R₂ of Formula 1 are palmitoyl and linoleoyl, respectively. The compound of Formula 2 is sometimes referred as “PLAG” or “EC-18” in this disclosure.

The monoacetyldiacylglycerol compounds can be separated and extracted from the natural deer antler or can be produced by known organic synthesis methods (Korean Patent No. 10-0789323). More specifically, deer antler is extracted with hexane, followed by extracting the residue with chloroform and removing the chloroform to provide chloroform extracts. The volume of the solvents for this extraction is just enough to immerse the deer antler. In general, about 4-5 liters of hexane and/or chloroform for 1 kg of deer antler is used, but not limited thereto. The extracts obtained by this method is further fractionated and purified using series of silica gel column chromatograph and TLC method to obtain the monoacetyldiacylglycerol compound of the present invention. A solvent for the extraction is selected among chloroform/methanol, hexane/ethylacetate/acetic acid, but not limited thereto.

A chemical synthetic method for the preparation of the monoacetyldiacylglycerol compounds is shown in Korean Patent No. 10-0789323. Specifically, the method comprises (a) a step of preparing 1-R1-3- protecting group—glycerol by introducing a protecting group in the position 3 of 1-R1-glycerol; (b) a step of preparing 1-R1-2-R2-3-protecting group-glycerol by introducing R2 in the position 2 of the 1-R1-3-protecting group—glycerol; and (c) a step of preparing the desired monoacetyldiacylglycerol compound by performing a deprotection reaction and the acetylation reaction of the 1-R1-3-protecting group—glycerol at the same time. The monoacetyldiacylglycerol compound may be further purified if necessary. Alternatively, monoacetyldiacylglycerol compounds can be prepared by acid decomposition of phosphatidylcholine(acetolysis) but is not limited thereto. Stereoisomers of the compounds of formula 1 are also within the scope of the invention.

The monoacetyldiacylglycerol compound of the present invention can be effectively used for the treatment and/or alleviation of diabetes. The term “diabetes” is a chronic disease resulting from abnormalities in glucose, lipid and/or amino acid metabolism due to failure of insulin function. Diabetes is largely classified into type I diabetes (insulin dependent diabetes mellitus: IDDM) in which β cells on the

Langerhans' island are destroyed and insulin secretion is irreversibly reduced to become hyperglycemia, and type II diabetes (non-insulin dependent diabetes mellitus: NIDDM) in which the insulin response to glucose decreases or insulin resistance to glucose increases in the Langerhans' island, and thereby resulting in chronic hyperglycemia. The monoacetyldiacylglycerol compound according to the present invention can be used for the treatment of the type I diabetes and the type II diabetes. The term “treatment” refers to any action by the composition in which symptoms caused by diabetes are improved or beneficially altered.

According to the embodiment of the present invention, when the monoacetyldiacylglycerol compound is administered, it was found that 1) the weight loss due to diabetes can be recovered to a normal state, 2) the insulin expression in pancreatic tissue can be increased (Example 1), and 3) the pancreatic beta cells damage can be reduced. These facts indicate that the administration of the monoacetyldiacylglycerol compound improves diabetes.

Glucose is one of body's main energy sources and is used by most cells and plays an important role in cell function. When the glucose is ingested, blood glucose levels increase to secrete insulin in pancreatic beta cells. In response to the secreted insulin, the blood glucose is absorbed into muscle or adipose tissue so as to be used as an energy source. However, the increased blood glucose levels have a detrimental effect on function and survival of pancreatic beta cells. The high concentration of glucose induces overstimulation of beta cells, which makes the insulin synthesis rate slower than the insulin secretion rate. As a result, glucose-stimulated insulin secretion (GSIS), the most important function of beta cells, does not work properly.

The body cells do not accept glucose by themselves, but they receive the glucose through a protein called glucose transporters (GLUTs). That is, after ingesting foods, the GLUTs transport the glucose in blood into cells. Among the various glucose transporters (GLUTs), glucose transporter 2 (GLUT2) and glucose transporter 4 (GLUT4) control the glucose uptake by insulin stimulation. The GLUT2 is closely related with the insulin resistance. If the GLUT2 does not work properly, the secretion of insulin in pancreatic beta cells does not occur normally. The reduced insulin secretion prevents many tissues from properly absorbing the blood glucose, and the high blood glucose concentration eventually produce adverse effect on the beta cell survival. Also, the high blood glucose concentration induces oxidative stress of cells and endoplasmic reticulum stress. The oxidative stress is also caused by glucose autooxidation, glycation product formation, glycosylation of proteins in diabetics and reactive oxygen species (ROS). Since the beta cells of the pancreas express antioxidant enzymes in low level, beta cells are destructed due to the oxidative stress, and cell differentiation is suppressed. In addition, the high blood glucose concentration caused by diabetes induces inflammatory cell formation. These inflammatory cells secrete cytokines and activate stress signaling pathways to inhibit and destroy pancreatic beta cell function.

In the embodiments of the present invention, the pancreatic tissues of subjects were investigated after various experiments. According to the investigations, when the monoacetyldiacylglycerol compound is administered, 1) the body weight loss which is caused by diabetes was not observed, and the insulin expression was increased in pancreatic tissue (Example 1), 2) the expression of apoptosis-related proteins was decreased in the pancreatic beta cell line INS-1 (Example 3), 3) the endocytosis of GLUT2 was promoted (Example 4), 4) the generation of ROS due to high glucose level in beta cells was reduced (Example 5), and 5) Glucose uptake of beta cells was regulated (Example 6). Thus, the monoacetyldiacylglycerol compounds prevent the excessive glucose uptake of the pancreatic beta cells, promote endocytosis of GLUT2 and maintain normal beta cell function, thereby alleviate detrimental effects caused by the rapid glucose uptake. As a result, it could be seen that the monoacetyldiacylglycerol compound is effective for the treatment of diabetes.

The ROS naturally occurs by the normal oxygen metabolism and plays an important role in cell signaling and homeostasis. The excessive glucose or excessively produced fructose binds to proteins to produce ROS, which induces apoptosis and diabetic complications.

In summary, because excessive glucose intake induces the ROS generation and oxidative stress, this should be controlled. The PLAG promotes endocytosis of GLUT2. Thus, even if the beta cells are exposed to high glucose level, PLAG regulates the rapid influx of glucose, and alleviates the damage of pancreatic beta cells due to the high glucose concentration. The undamaged beta cells normally secrete insulin, and the blood glucose is absorbed into fat and muscle tissue. Thus, PLAG can prevent pancreatic beta cells damage which is caused by diabetes and hyperglycemia.

The pharmaceutical composition comprising the monoacetyldiacylglycerol compound of the present invention may include conventional pharmaceutically acceptable carriers, excipients, or diluents. The amount of monoacetyldiacylglycerol in the pharmaceutical composition can be widely varied without specific limitation, and is specifically 0.0001 to 100 weight %, preferably, 0.001 to 90 weight %, for example, the monoacetyldiacylglycerol may be contained in 70 to 80 weight %, with respect to the total amount of the composition.

Additionally, in one aspect, one or more therapeutic compound compounds of Formula 1 or 2, or PLAG and one or more distinct diabetic medications or diabetic treating agents can be administered in combination to a patient.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to diabetes. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

The administration of the compounds (e.g., compounds of Formula 1 or 2, or PLAG) and the one or more diabetic medications or diabetic treating agents for Type 1 diabetes, Type 2 diabetes, prediabetes, and gestational diabetes.

The compound (e.g., compounds of Formula 1 or 2, or PLAG) and the one or more diabetic medications or diabetic treating agents may be administered simultaneously or sequentially. In some embodiments, the diabetes treatment is an established therapy for the disease indication and by addition of compounds (e.g., compounds of Formula 1 or 2, or PLAG), such treatment improves the therapeutic benefit to the patients. Such improvement could be measured as increased responses on a per patient basis or increased responses in the patient population. Combination therapy could also provide improved responses at lower or less frequent doses of therapeutic agents resulting in a better tolerated treatment regimen. As indicated, the combined therapy of one or more compounds of Formula 1 or 2, or PLAG and one or more distinct medications for treating diabetes or diabetic treating agents can enhance clinical activity, for example, by i) administering insulin; ii) administering agents that increase the amount of insulin secreted by the pancreas, iii) administering agents that increase the sensitivity of target organs to insulin, and/or iv) administering agents that decrease the rate at which glucose is absorbed from the gastrointestinal tract.

In some embodiments, the methods (e.g., combination therapy for diabetes treatment) may include administration of a second, distinct therapeutic agent (e.g., diabetic medications or diabetic treating agents) or treatment with a second therapy (e.g., a therapeutic agent or therapy that is standard in the art) for diabetes treatment.

In some embodiments, the methods (e.g., combination therapy for diabetes treatment) may include administration of a second therapeutic agent (e.g., diabetic medications or diabetic treating agents) or treatment with a second therapy (e.g., a therapeutic agent or therapy that is standard in the art) for treatment of diabetes (e.g., Type 1 diabetes, Type 2 diabetes, prediabetes, and gestational diabetes).

Exemplary therapeutic agents include one or more diabetic medications or diabetic treating agents. A “diabetic medication” or “diabetic treating agent” or other similar term is a chemical compound (drug) or biologis useful in the treatment of diabetes (e.g., Type 1 diabetes, Type 2 diabetes, prediabetes, and gestational diabetes) by controlling blood sugar (glucose) level. Generally, a “diabetic medication” or “diabetic treating agent” as referred to herein will be distinct from a compound of Formulae 1 or 2, such as PLAG.

Examples of diabetic medications or diabetic treating agents that may be issued in methods, kits and compositoons herein, including in combination or conjunction with administration of a compound of Formulae 1 or 2 such as PLAG include, but not are limited to, an insulin or insulins (e.g., Humulin, Novolin, NovoLog, FlexPen, Fiasp, Apidra, Humalog, Humulin N, Novolin N, Tresiba, Levemir, Lantus, Toujeo, NovoLog Mix 70/30, Humalog Mix 75/25, Humalog Mix 50/50, Humulin 70/30, Novolin 70/30, Ryzodeg, and the like), amylinomimetic drug or pramlintide (e.g., SymlinPen 120 and SymlinPen 60), alpha-glucosidase inhibitors (e.g., acarbose (Precose), and miglito1(Glyset)), biguanides (e.g., metformin-alogliptin (Kazano), metformin-canagliflozin (Invokamet), metformin-dapagliflozin (Xigduo XR), metformin-empagliflozin (Synjardy), metformin-glipizide, metformin-glyburide (Glucovance), metformin-linagliptin (Jentadueto), metformin-pioglitazone (Actopius), metformin-repaglinide (PrandiMet), metformin-rosiglitazone (Avand.amet), metformin-saxagliptin (Kombiglyze XR), and metformin-sitagliptin (Janumet)), dopamine agonist (e.g., Bromocriptine (Cycloset)), dipeptidyl peptidase-4 (DPP-4) inhibitors (e.g., alogliptin (Nesina), alogliptin-metformin (Kazano), alogliptin-pioglitazone (Oseni), linagliptin (Tradjenta), tinagliptin-empagliflozin (Glyxambi), linagliptin-metformin (Jentadueto), saxagliptin (Onglyza), saxagliptin-metformin (Kotnbiglyze XR), sitagliptin (Januvia), sitagliptin-metformin (Janumet and Janumet XR), sitagliptin and simvastatin (Juvisync)), glucagon-like peptide-1 receptor agonists (e.g., aibigiutide (Tanzeum), dulaglutide (Trulicity), exenatide (Byetta), exenatide extended-release (Bydureon), liraglutide (Victoza), and semagiutide (Ozempie)), meglitinides (e.g,, nateglinide (Starlix), repaglinide (Prandin), and repaglinide-metformin (Prandimet)), sodium-glucose transporter (SGLT) 2 inhibitors (e.g., dapagliflozin (Farxiga), dapagliftozin-metformin (Xigduo XR), eanagliflozin (Invokana), canagliflozin-metformin (Invokamet), empagliflozin (Jardiance), empagliflozin-linagliptin (Glyxambi), empagliflozin-metfomin (Synjardy), and ertugliflozin (Stegiatro)), sulfonylureas (e.g., glimepiride (Amaryl), glimepiride-pioglitazone (Duetact), glimepiride-rosiditazone (Avandaryl), gliclazide, glipizide (Ohicotrol), glipizide-metformin (Metaglip), glyburide (DiaBeta, Glynase, Micronase), glyburide-metformin (Glucovance), chlorpropamide (Diabinese), tolazamide (Tolinase), and tolbutamide (Orinase, Tot-Tab)), thiazolidinediones (e.g., rosiglitazone (Avandia), rosiglitazone-glitnepiride (Avandaryl), rosiditazone-rnetform ill (Amaryl M), pioglitazone (Actor), pioghtazone-alogliptin (Oseni), pioglitazone-glimepiride (Duetact), and pioglitazone-metformin (Actoplus Met, Actoplus Met XR)), asprin and the like, and pharmaceutically acceptable salts, or acids of any of the above.

Exemplary effective daily doses of the diabetic medications or diabetic treating agents include, for agents that are in current clinical use, the dosages that the agents are currently administered. In general, suitable or exemplary effective daily doses of the diabetic medications or diabetic treating agents general may be between 0.1 μg/kg and 100 μg/kg body weight, e.g., 0.1, 0.3, 0.5, 1, 5, 10, 15, 20, 25, 30, 35, 40, 45, 50, 55, 60, 65, 70, 75, 80, 85, 90, 95, or 99 μg/kg body weight.

Alternatively, the distinct one or more diabetic medications or diabetic treating agents may be administered about once per week, e.g., about once every 7 days. Or, the distinct one or more diabetic medications or diabetic treating agents suitably may be administered twice per week, three times per week, four times per week, five times per week, six times per week, or seven times per week. Exemplary effective weekly doses of the one or more diabetic medications or diabetic treating agents include between 0.0001 mg/kg and 4 mg/kg body weight, e.g., 0.001, 0.003, 0.005, 0.01. 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, or 4 mg/kg body weight. For example, an effective weekly dose of the distinct one or more diabetic medications or diabetic treating agents s between 0.1 ₗig /kg body weight and 400 μg/kg body weight of a patient.

The pharmaceutical composition of the present invention may further include other active ingredients having a therapeutic effect of diabetes. The pharmaceutical composition may be formulated into solid, liquid, gel or suspension form for oral or non-oral administration, for example, tablet, bolus, powder, granule, capsule such as hard or soft gelatin capsule, emulsion, suspension, syrup, emulsifiable concentrate, sterilized aqueous solution, non-aqueous solution, freeze-dried formulation, and so on. In formulating the composition, conventional excipients or diluents such as fillers, bulking agents, binders, wetting agents, disintegrating agents, and surfactants can be used. The solid formulation for oral administration includes tablet, bolus, powder, granule, capsule and so on, and the solid formulation can be prepared by mixing one or more of the active components and at least one excipient such as starch, calcium carbonate, sucrose, lactose, gelatin, and so on. Besides the excipient, a lubricant such as Magnesium stearate and talc can also be used. The liquid formulation for oral administration includes emulsion, suspension, syrup, and so on, and may include conventional diluents such as water and liquid paraffin or may include various excipients such as wetting agents, sweeting agents, flavoring agents, and preserving agents. The formulation for non-oral administration includes sterilized aqueous solution, non-aqueous solution, freeze-dried formulation, suppository, and so on, and solvent for such solution may include propylene glycol, polyethylene glycol, vegetable oils such as olive oil, and ester for syringe injection such as ethyl oleate. Base materials of the suppository may include witepsol, macrogol, tween 61, cacao butter, Laurin and glycerogelatin.

The monoacetyldiacylglycerol compound can be administered in a pharmaceutically effective amount. The term “pharmaceutically effective amount” is used to refer to an amount that is sufficient to achieve a desired result in a medical treatment. The “pharmaceutically effective amount” can be determined according to the subject's category, age, sex, severity and type of disease, activity of drug, sensitivity to drug, administration time, administration route, excretion rate, and so forth. The composition of the present invention can be administered alone or with other therapeutic agents sequentially or simultaneously. The composition of the present invention can be administered once or multiple times. The preferable amount of the composition of the present invention can be varied according to the condition and weight of patient, severity of disease, formulation type of drug, administration route and period of treatment. An appropriate total amount of administration of a compound of Formulae 1 or 2 such as PLAG per 1 day can be determined by a physician and is generally about 0.001 to about 5,000 mg/kg, preferably about 0.05 to 1,000 mg/kg, once a day or can be administered in divided doses multiple times daily. The composition of the present invention can be administered to any subject that requires the prevention or treatment of diabetes. For example, the composition of the present invention can be administered to not only human but also non-human animal (specifically mammals) such as monkey, dog, cat, rabbit, guinea pig, rat, mouse, cow, sheep, pig, goat, and so on.

In some embodiments, the present invention provides a health functional food composition for preventing, alleviating or improving diabetes, which comprises a monoacetyldiacylglycerol of formula 1 as an active ingredient.

The monoacetyldiacylglycerol compound according to the present invention may be included into a health functional food composition to improve diabetes in a subject. The monoacetyldiacylglycerol compound and the diabetes disease are as described above. When the compound of the present invention is included into the health functional food composition, the amount of monoacetyldiacylglycerol in the health food composition can be determined suitably according to the intended use. Generally, the amount of monoacetyldiacylglycerol is preferably from 0.01 to less than 15 weight %, with respect to the total amount of the health functional food composition when the monoacetyldiacylglycerol is included in food or beverages. However, the amount of monoacetyldiacylglycerol may be increased or decreased. In case of long term use for the purpose of the health control and hygiene, the amount of the monoacetyldiacylglycerol can be less than the above range. Since there is no problem in terms of safety, the monoacetyldiacylglycerol may be used in an amount greater than the above range. Foods to which the compound of the present invention can be added are not limited and include various foods, for example, meats, sausages, breads, chocolates, candies, snacks, pizzas, noodles, gums, daily products such as ice creams, soups, beverages, teas, drinks, alcoholic beverages, vitamin complexes and any health functional food.

When the monoacetyldiacylglycerol is used in the beverage product, the beverage product may include sweeting agents, flavoring agents or carbohydrates. Examples of carbohydrates include monosaccharides such as glucose and fructose, disaccharides such as maltose and sucrose, polysaccharides such as dextrin and cyclodextrin, and sugar alcohols such as xylitol, sorbitol and erythritol. The amount of carbohydrate in the beverage composition can be widely varied without specific limitation, and is preferably 0.01 to 0.04 g, more preferably, 0.02 to 0.03 g per 100 ml of the beverage. Examples of sweeting agents include natural sweeteners such as thaumatin and stevia extract and artificial sweeteners such as saccharin and aspartame. In addition to the above, the health functional food composition of the present invention may include various nutrients, vitamins, electrolytes, flavoring agents, colorants, pectic acid and salts thereof, alginic acid and salts thereof, organic acids, protective colloidal thickening agents, pH controlling agents, stabilizing agents, preserving agents, glycerin, alcohol, carbonizing agents used in carbonated beverages and so on. Moreover, the health functional food composition of the present invention may include fruits, as used in preparing natural fruit juices and fruit juice beverages and vegetable beverages.

The present invention provides a method for treating diabetes, comprising the step of administering the pharmaceutical composition to a suspected individual of diabetes disease. By administering the composition to a suspected subject of diabetes, the diabetes is efficiently treated. The term “suspected subject of diabetes” refer to those that have or are likely to develop diabetes disease. The diabetes disease can be treated or prevented by administering an effective amount of the compound to a patient in need thereof. The kind of the monoacetyldiacylglycerol compound and the dose of the monoacetyldiacylglycerol compound and the diabetes disease are as described above. The term “administration” means introducing the pharmaceutical composition of the present invention to a patient in need by any suitable method. The route of administration may be any or a various routes, oral or non-oral, as long as the target tissue can be reached, for example, oral administration, intraperitoneal administration, transdermal administration(topical application, etc.), intravenous administration, intramuscular administration, subcutaneous administration, intradermal administration, intranasal administration, rectal administration, intranasal administration, intraperitoneal administration and the like may be used, but is not limited thereto.

### EXAMPLES

The following examples are provided for better understanding of this invention. However, the present invention is not limited by the examples.

In order to confirm the efficacy of 1-palmitoyl-2-linoleoyl-3- acetyl-racglycerol (EC-18 or PLAG) in the treatment of diabetes disease, Streptozotocin (STZ)-induced diabetes model were used in the experiments.

### Experimental Example: Preparation of Control and Experimental Groups

Mice were divided into four groups (control group, STZ-only treatment group, PLAG-co-treatment group, and PLAG-post-treatment group). After a 16-h fast, the three treated groups except the control group were injected intraperitoneally with STZ (200 mg/kg BW) prepared fresh in citrate buffer, wherein BW means body weight. STZ-only treatment mice received no additional treatment.

On the same day, PLAG-co-treatment group mice began treatment with PLAG (250 mg/kg, p.o.) once daily for 3 consecutive days. The PLAG-post-treatment group received PLAG (250 mg/kg, p.o.) for 2 consecutive days beginning 1 day after STZ injection. As the PLAG, 1-palmitoyl-2-linoleoyl-3-acetyl-rac-glycerol represented by Formula 2 was used.

### Example 1: Blood glucose and weight change measurement

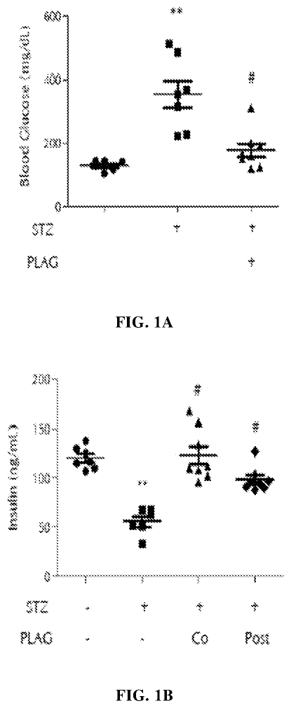

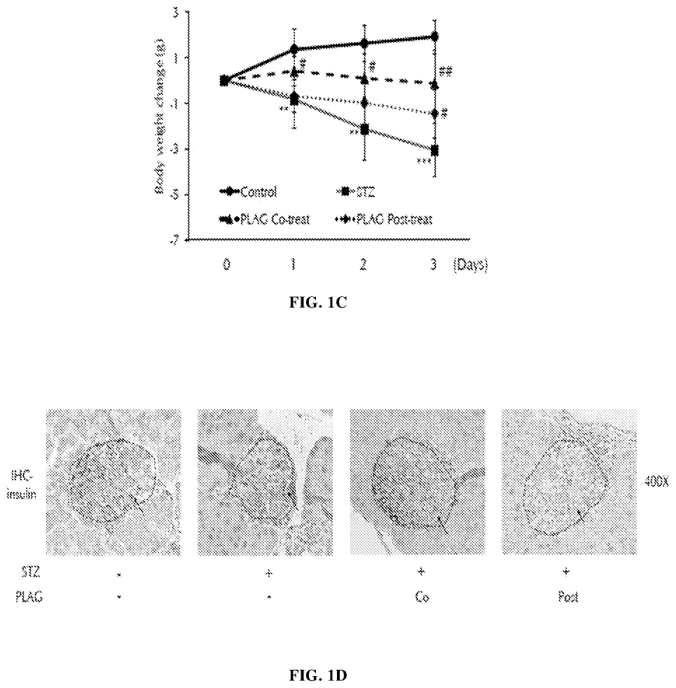

Blood was collected via the retro-orbital plexus, and blood glucose levels were monitored during the experiment. Blood glucose was measured using the Accu-Chek glucometer (Roche, Seoul, Republic of Korea). FIG. 1A is a chart measuring the blood glucose of the control group and the experimental groups on the first day.

All mice were sacrificed on day 4, last day of experiment (day 4), serum insulin was measured (FIG. 1B). Tissues of the pancreatic tissue of the control group and the experimental group were collected and fixed in 10% formalin for further analysis. During the experiment, the weight change of the control group and the experimental groups was measured (FIG. 1C).

Referring to FIG. 1A to 1C, the first day, the blood glucose increase was observed in STZ-only treated animals. However, the PLAG-co-treatment group did not have a significant increase in blood glucose compared with the control group. Insulin secretion of the STZ-only treatment group is significantly lower than the control and other experimental groups. Also the STZ-only treatment group has a greater weight change than the control and other experimental groups.

### Example 2: Pancreas Islet Histopathology

Pancreas tissues were fixed in 10% formalin, embedded in paraffin, and sectioned at a 4 μm thickness. For immunohistochemistry, sections were deparaffinized and dehydrated using xylene and a graded ethanol series Staining was performed using the Real EnVision Detection System Peroxidase-DAB kit (Dako, Glostrup, Denmark) following the manufacturer's instructions, and then observed under a light microscope (Olympus, Tokyo, Japan) (FIG. 1D, 400 magnification).

### Example 3: Effect of PLAG on Cell Death

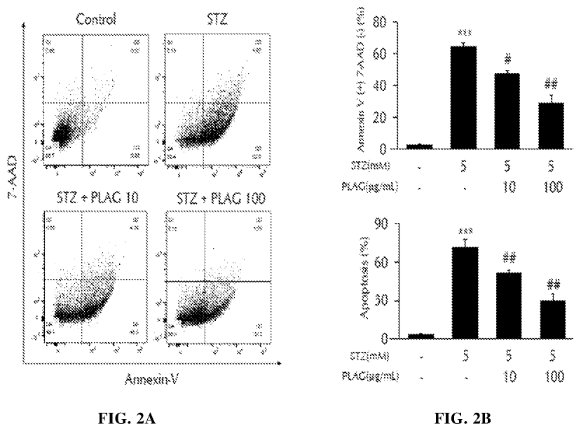

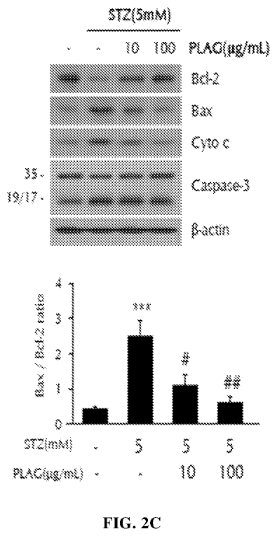

The effect of PLAG on STZ-induced cell apoptosis was analyzed using flow cytometry using annexin-V and 7-AAD dye (FIGS. 2A and 2B). Cells were collected by trypsinization and washed with PBS. For cell apoptosis analyses, INS-1 cells were incubated with annexin V (BD Biosciences, Franklin Lakes, N.J., USA) for 10 min at room temperature, and stained with 7-AAD (BD Biosciences). Analysis was performed using a BD FACSVerse flow cytometer (BD Biosciences). The annexin-V and 7-AAD dyes are dyes that label apoptosis, one kind of cell death.

In addition, to perform a special protein detection test (Western Blotting), cells were lysed by RIPA buffer (LPS Solution, Daejeon, Korea) supplemented with protease and phosphatase inhibitors (Thermo Scientific, Waltham, Mass., USA). Proteins were separated on 12% sodium dodecyl sulfate-polyacrylamide gels and transferred to polyvinylidene difluoride membranes (EMD Millipore, Darmstadt, Germany). The membranes were blocked with 5% BSA for 1 h and incubated with primary antibodies to GLUT2 (bs-0351r, Bioss, Woburn, Mass., USA), RAC1 (03589, EMD Millipore), BAX (B51030, Bioworld Tech, St. Louis Park, Minn., USA), BCL-2 (B51031, Bioworld), Cytochrome c (#4272, Cell Signaling Technology, Danvers, Mass., USA), Caspase-3 (#9662, Cell Signaling Technology), and Na+-K+ ATPase (#3010S, Cell Signaling Technology). After three washes in PBST, membranes were incubated with HRP-conjugated secondary antibodies (Enzo Life Sciences, dilution 1:5,000) for 1 h at room temperature. Protein bands were detected using ECL reagent (Thermo Scientific) and then visualized on films. Expression of apoptosis-related proteins like Bcl-2, Bax, cytochrome c, caspase-3 was analyzed by western blotting, Bax/Bcl-2 ratio is represented by a bar graph (FIG. 2-C). Data are presented as mean ±SD (***P<0.001 vs. control, #P<0.05, ##P<0.005 vs. STZ).

Referring FIGS. 2A to 2C, in cells treated with 10 μg/mL of PLAG, apoptosis was observed in ˜50%, and it was ˜30% in the 100 μg/mL PLAG-treatment group, indicating dose-dependent protection.

The anti-apoptotic protein BCL-2 was decreased by STZ and recovered by PLAG. Conversely, expression of the apoptosis-related proteins BAX, cytochrome c, and caspase-3 were increased by STZ and attenuated by PLAG addition.

### Example 4: Effect of PLAG on GLUT2 Expression in Cell Membranes

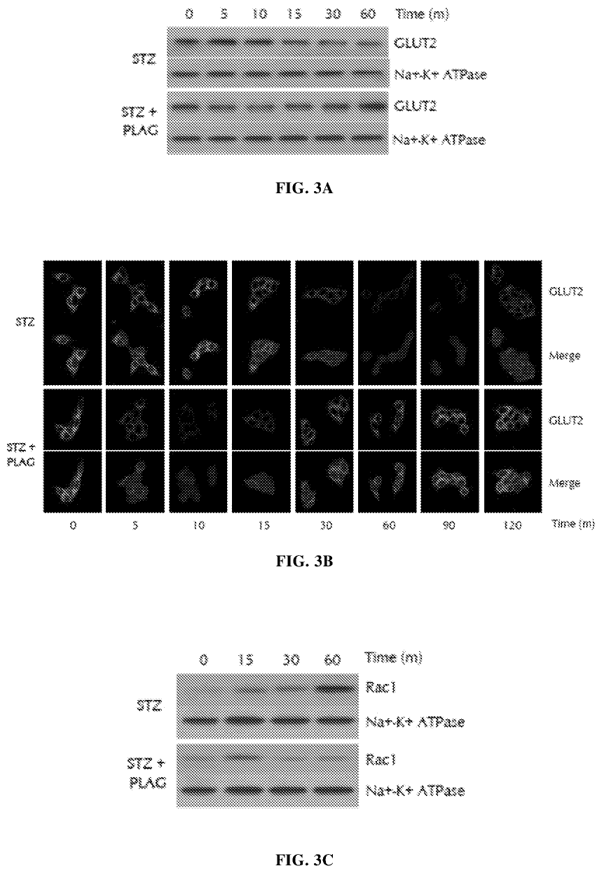

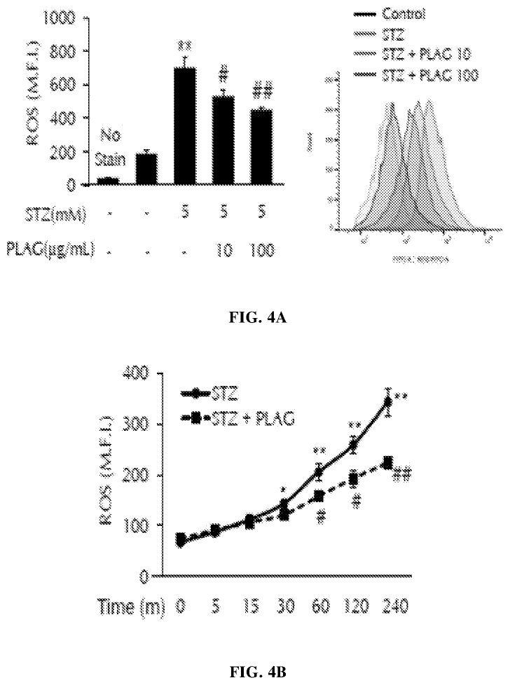

To investigate the effect of PLAG on GLUT2 plasma membrane localization, GLUT2 expression and Racl expression in membrane fractions were examined in the same manner as the Western protein blotting (FIGS. 3A to 3B).

In addition, the localization of GLUT2 was observed by immunofluorescence assay. Cells were grown on glass coverslips in 24-well plates and treated with STZ and PLAG. After washing with ice-cold PBS, the cells were fixed with 4% formaldehyde. Permeabilization was not performed to identify only proteins expressed in the cell membrane. The cells were incubated with anti-GLUT2 antibodies (dilution 1:500) and then stained with an Alexa Fluor 488-conjugated secondary antibody (Enzo Life Sciences, dilution 1:1000) and DAPI (Invitrogen). Stained cells were observed under a Zeiss LSM800 confocal microscope (Carl Zeiss, Jena, Germany) (FIG. 3-C).

Referring to FIG. 3A, membrane-expressed GLUT2 steadily decreased in STZ-treated cells. In PLAG-treated cells, GLUT2 expression gradually decreased until 10 min, then recovered and returned to control levels at 60 min

Referring to FIG. 3B, the expression of RAC1, an NADPH oxidase that produces ROS, steadily increased in membrane fractions in the STZ group, but in the PLAG-treated group it was attenuated after a slight increase in 15 min. Referring to FIG. 3-C, in PLAG-treated cells, accelerated GLUT2 internalization was observed, and GLUT2 was again observed in the membrane at 60 min

### Example 5: Effect of PLAG on ROS

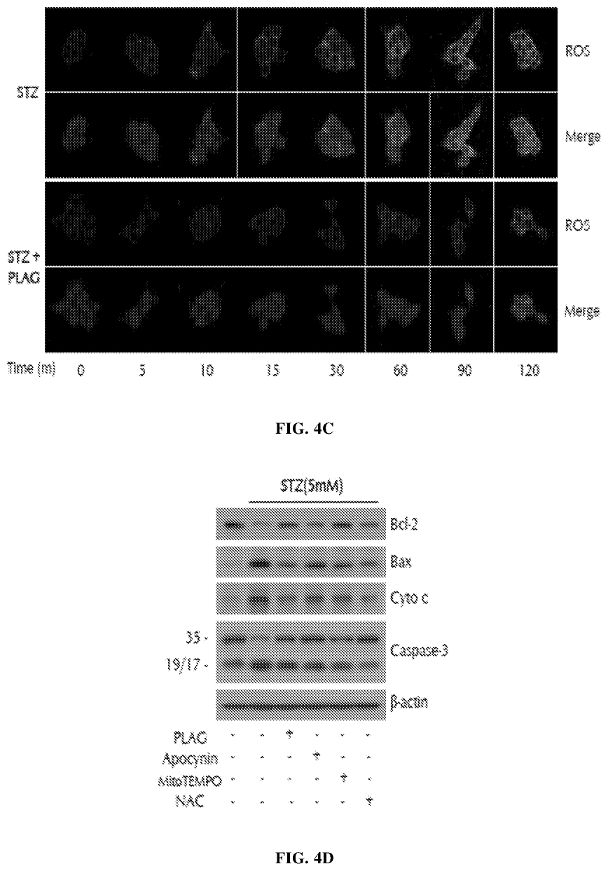

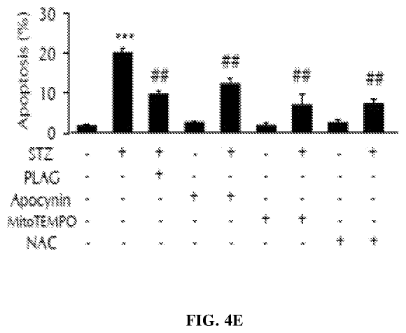

For intracellular ROS analyses after administering PLAG, cells were incubated with 2 μM of DCFH-DA (2′,7′-dichlorodihydrofluorescein diacetate, Invitrogen, Carlsbad, CA, USA) for 30 min at 37 ° C. The analysis was performed using a BD FACS Verse flow cytometer (BD Biosciences) (FIGS. 4A and 4B), and expression of reactive oxygen species (ROS) was observed by immunofluorescence assay (FIG. 4C). We further examined cell apoptosis in cells co-treated with three types of ROS inhibitor (Apocynin, MitoTEMPO, NAC) to examine the association of intracellular ROS generation with pancreatic beta cell apoptosis. Expressed apoptosis-related proteins and apoptosis degree were observed in inhibitor-treated cells (FIGS. 4D and 4E). Apocynin (NADPH oxidase inhibitor), MitoTEMPO (mitochondrial ROS inhibitor), and N-acetyl-L-cysteine (NAC, an overall ROS inhibitor) were used as the ROS inhibitor. Data are presented as mean ±SD(* P<0.05, ** P<0.005, *** P <0.001 vs control, #P<0.05, ## P<0.005 vs STZ).

Referring to FIG. 4A, intracellular ROS was increased in the STZ-treated cells and dose-dependently decreased in PLAG-treated cells. Referring to FIGS. 4B and 4C, ROS rapidly increased in a time-dependent fashion in STZ-treated cells, but this was attenuated in PLAG-treated cells.

Also, referring to FIGS. 4D and 4E, significant reductions of expressed apoptosis-related proteins and STZ-induced cell apoptosis were observed in inhibitor-treated cells. These results suggest that ROS generation following STZ treatment contributes to pancreatic beta cell apoptosis.

### Example 6: Effect of PLAG on Glucose Uptake of Beta Cells

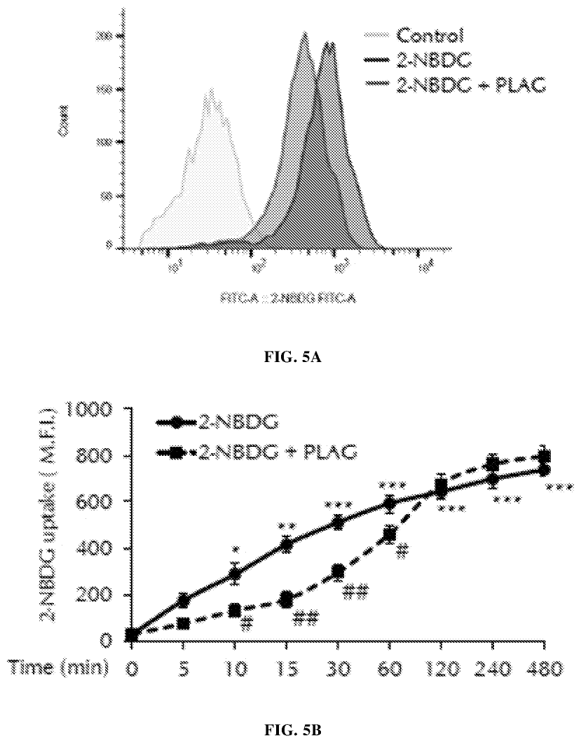

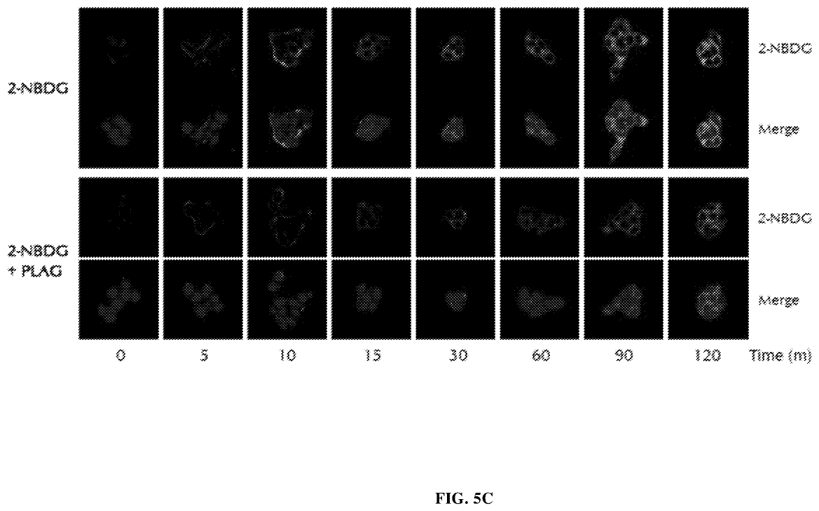

2-[N-(7-nitrobenz-2-oxa-1,3-diazol-4-yl)amino]-2-deoxy-D-glucose (2-NBDG, N13195, Thermo Scientific) was used for glucose uptake assays. Glucose uptake assays were performed using 2-NBDG conjugated with fluorescent dye. Intracellular 2-NBDG was measured at 60 min (FIG. 5A) and successively calculated from 5 min to 480 min after 2-NBDG treatment by How cytometry (FIG. 5B). Serum-containing complete medium was removed, and INS-1 cells were washed in PBS. The cells were cultured in glucose-free culture media for 1 h at 37° C. and then treated with 2-NBDG. Intracellular fluorescence was measured using a BD FACS Verse flow cytometer (BD Biosciences).

Intracellular 2-NBDG expression were also observed using immunofluorescence assay (FIG.5-C). Data are presented as mean ±SD. *P<0.05, **P<0.005, ***P<0.001 vs. control, #P<0.05, ##P<0.005 vs. STZ.

Referring to FIG. 5A, fluorescence intensity was measured in 2-NBDG-treated cells, but lower fluorescence intensity was detected in the PLAG-treated group at 60 min.

Referring to FIG. 5B, glucose uptake was attenuated in the experimental group administered PLAG rather than 2-NBDG alone. These results suggest that PLAG accelerates the internalization of GLUT2 and limits glucose influx.

Referring to FIG. 5C, in the 2-NBDG-only treated group, 2-NBDG was observed at the cell membrane at 5 min and gradually increased in the cytoplasm, and fluorescence intensity increased in a time-dependent manner Intracellular 2-NBDG increased more slowly in PLAG-treated groups. These results suggest that PLAG may attenuate the deleterious effects of rapid glucose uptake through promoting GLUT2 endocytosis and simultaneously maintaining normal beta cell function.

### Example 7: Effect of PLAG on GLUT2-Silenced Cells

GLUT2-silenced cells were prepared to clarify the role of GLUT2 in STZ-treated cells. INS-1 cells were transfected with GLUT2 siRNA to determine whether GLUT2 expression is related to STZ-induced cell apoptosis and ROS generation.

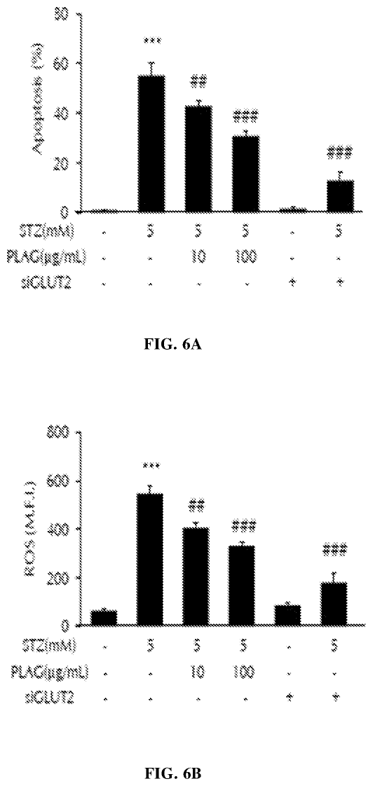

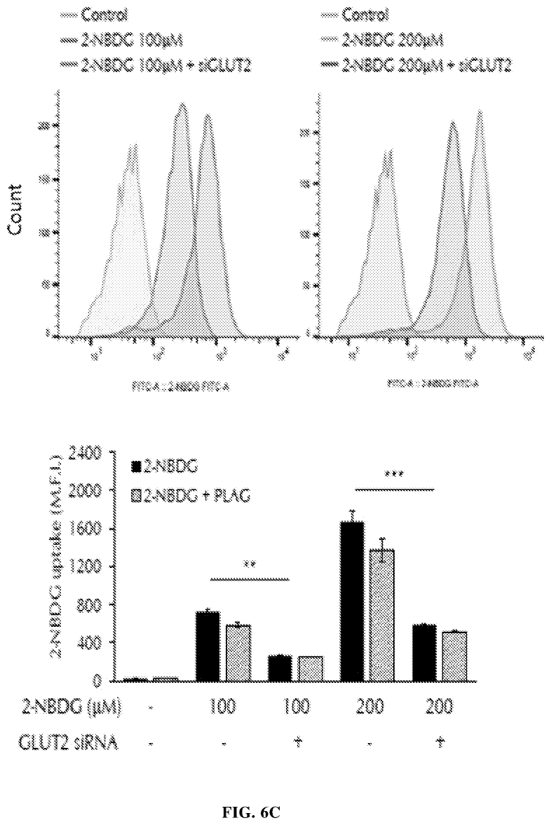

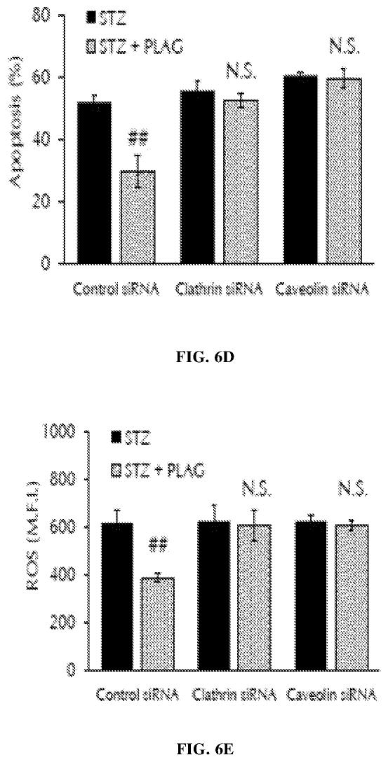

Apoptosis of pancreatic beta cells, intracellular ROS generation and glucose intake was analyzed by flow cytometry using GLUT2 siRNA transfected cells (FIGS. 6-A to 6-C). The effect of PLAG on (D) cell apoptosis and (E) ROS generation in Endocytosis-related protein, clathrin or caveolin siRNA-transfected cells was analyzed (FIG. 6D and 6-E). Data are presented as mean ±SD. **P<0.005, ***P<0.001 vs. control, ##P<0.005, #IIIIP<0.001 vs. STZ group. N.S.: No significance. Transfection with siRNAs was performed using the HiPerFect reagent (Qiagen, Hilden, Germany), according to the manufacturer's protocol. Specific siRNAs directed against GLUT2, clathrin, and caveolin were obtained from Santa Cruz Biotechnology (Dallas, Tex., USA).

Cell apoptosis (FIG. 6A) and intracellular ROS generation (FIG. 6B) were noticeably increased in STZ-treated but not GLUT2-silenced cells. Glucose uptake was also not significantly increased in GLUT2-silenced cells (FIG. 6-C). To determine if PLAG biological activity was dependent on intracellular trafficking of GLUT2, we silenced the expression of the endocytosis-related proteins clathrin and caveolin using siRNAs (FIGS. 6-D,6-E). PLAG did not influence apoptosis or ROS generation in clathrin- or caveolin-silenced cells. This suggests that action of PLAG is associated with endocytosis of GLUT2.

### Example 8: Comparison of the Effects of PLAG and PLH (Specificity of PLAG Activity).

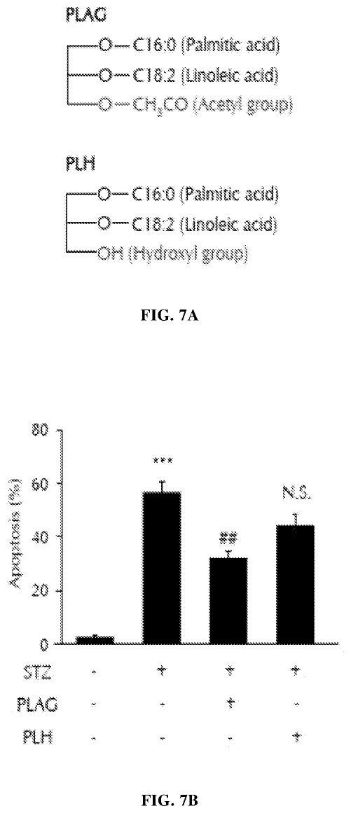

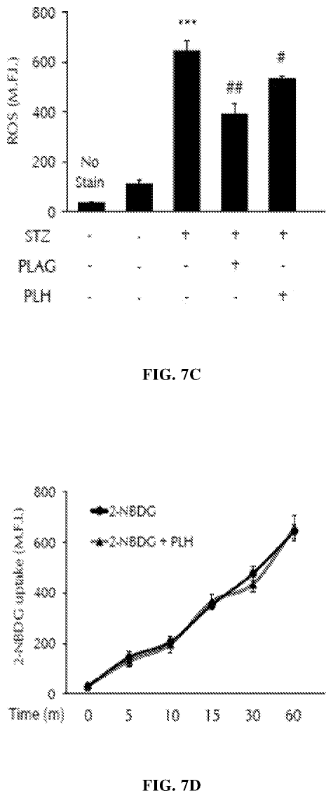

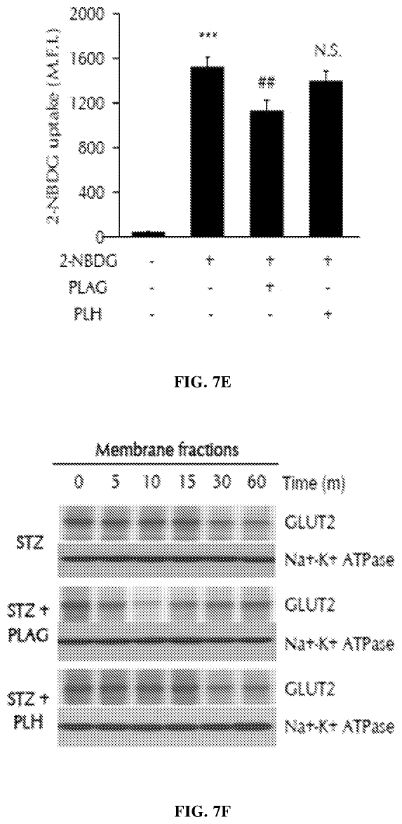

After treatment with PLAG and PLH(1-palmitoyl-2-linoleoyl-3-hydroxyl-rac-glycerol), apoptosis, intracellular ROS generation and glucose uptake were analyzed by flow cytometry (FIG. 7-B to 7-E). In addition, expression of GLUT2 in membrane fractions was analyzed by Western blotting (FIG.7-F). Data are presented as mean ±SD. ***P<0.001 vs. control, #P<0.05, ##P<0.005 vs. STZ. N.S.: No significance.

FIG. 7A shows simple structures of PLAG and PLH. PLH is a structural analogue of PLAG. PLAG has an acetyl group, but PLH has a hydroxyl group at the 3-position of glycerol. Referring to FIG. 7-B and 7-C, PLAG was more effective than PLH in decreasing cell apoptosis and ROS generation. In glucose uptake assays, the PLH-treated group showed a similar pattern to the control group and there was no effect on glucose uptake in PLH-treated cells. In the experimental group administered with PLAG, 2-NBDG was slowly absorbed and endocytosis of GLUT2 was promoted. Conversely, there was no change in 2-NBDG uptake or GLUT2 internalization in PLH-treated cells (FIG. 7D and 7E). These results confirm the specificity of PLAG in promoting GLUT2 endocytosis.

### Example 9: Effects of PLAG in High Glucose (HG)-Induced Cells

### PLAG Reduces HG-Induced Cell Apoptosis (FIG. 8)

Effect of PLAG on pancreatic beta cell protection was investigated after HG-treatment. HG-induced cell apoptosis was analyzed using flow cytometry. INS-1 cells were pre-treated with 50 or 100 μg/mL of PLAG for lh, and then treated with 30 mM of HG for 48h. When INS-1 cells were maintained in a high glucose conditions, there was a significant increase in cell apoptosis compared to control cells. In contrast, a decreased rate of apoptosis was observed in the cells treated with 50 μg/mL or 100 μg/mL of PLAG, indicating dose-dependent protection.

The protective effect of PLAG on glucotoxicity-induced pancreatic beta cell damage was further investigated after HG treatment. Cell apoptosis was increased up to 35% in HG-treated cells, and PLAG dose-dependently decreased the HG-induced cell apoptosis.

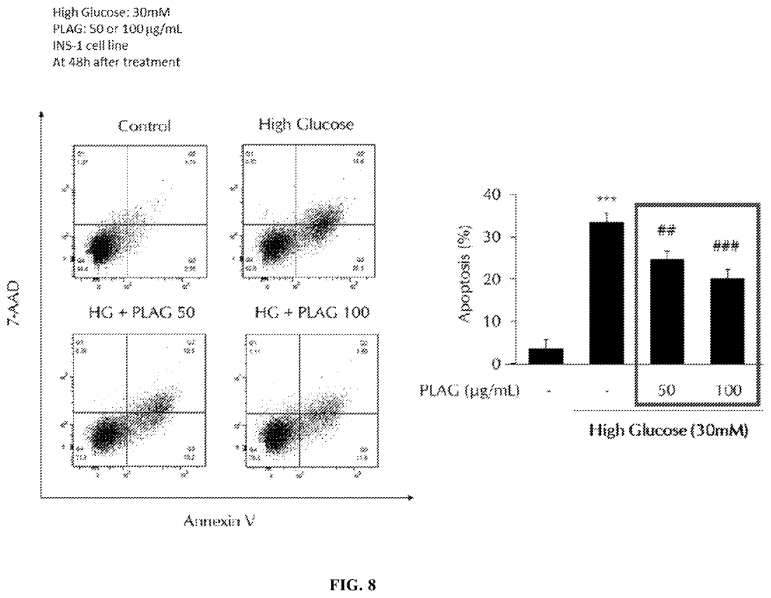

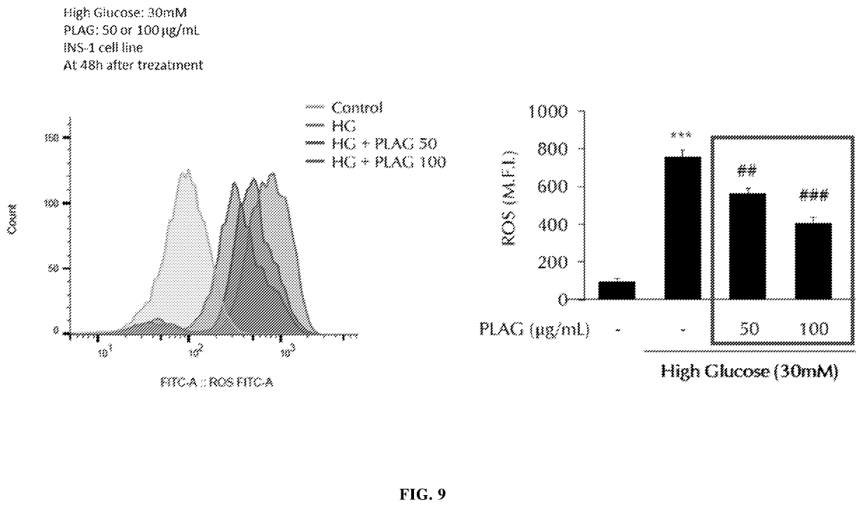

PLAG reduces HG-induced intracellular ROS generation (FIG. 9)

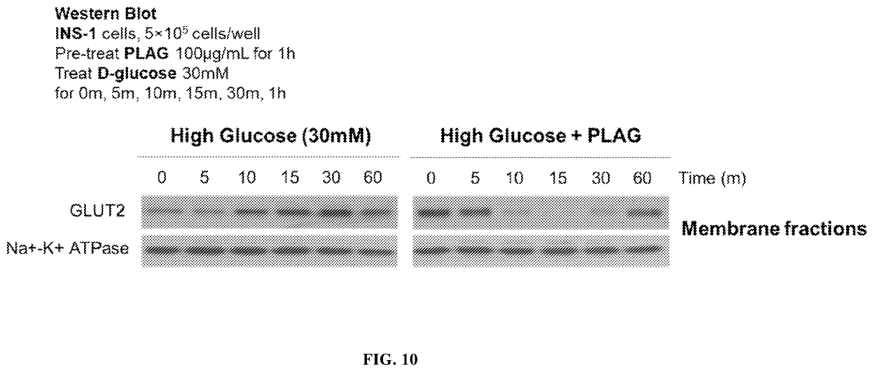

Intracellular ROS generation also analyzed by flow cytometry using DCFH-DA dye. ROS generation was determined by analyzing changes in fluorescence intensity. INS-1 cells were pre-treated with 50 or 100 μg/mL of PLAG for 1 h, and then treated with 30 mM of HG for 48h. INS-1 cells treated with 30mM HG showed significantly enhanced fluorescence intensity, and PLAG treatment dose-dependently decreased the HG-induced intracellular ROS generation. PLAG promotes GLUT2 endocytosis in HG-treated INS-1 cells (FIG. 10)

To investigate the effect of PLAG on GLUT2 plasma membrane localization, membrane proteins were fractionated and analyzed by western blotting. INS-1 cells were pre-treated with 100 pg/mL of PLAG for lh, and then treated with 30 mM of HG for indicating times. Membrane-expressed GLUT2 steadily increased in HG-treated cells. In PLAG-treated cells, GLUT2 expression was gradually decreased until 15 min, then recovered and returned to control levels at 60 min These results suggest that PLAG promotes GLUT2 internalization in high glucose conditions.

MATERIALS AND METHODS

### Cell Culture

INS-1 rat insulinoma pancreas beta cells were cultured in RPMI-1640 medium (Welgene, Gyeongsangbuk-do, Republic of Korea) containing 10% fetal bovine serum (Tissue Culture Biologicals, Long Beach, Calif., USA), 50 μM β-mercaptoethanol, 100 units/mL penicillin, and 100 μg/mL streptomycin (Antibiotic-Antimycotic Solution, Welgene). The cells were grown in a humidified atmosphere with 5% CO2 at 37° C. Chemicals and reagents

PLAG was obtained from Enzychem Lifesciences (Seoul, Republic of Korea). D-glucose was purchased from Sigma-Aldrich (St. Louis, Mo., USA). PLAG was dissolved in ethanol and the final working concentration was 0.1% (v/v). D-glucose was dissolved in cell culture medium.

### Flow Cytometry

Cells were collected by trypsinization and washed with ice-cold PBS. For cell apoptosis analyses, INS-1 cells were incubated with annexin V (BD Biosciences, Franklin Lakes, N.J., USA) for 10 min at room temperature, and stained with 7-AAD (BD Biosciences). For intracellular reactive oxygen species (ROS) analyses, cells were incubated with 2 μM of DCFH-DA (2′,7′-dichlorodihydrofluorescein diacetate, Invitrogen, Carlsbad, Calif., USA) for 30 min at 37° C. The analysis was performed using a BD FACS Verse flow cytometer (BD Biosciences).

### Western Blotting

For membrane protein fractionation, we performed using the MemPER™ Plus Kit (Thermo Scientific, Waltham, Mass., USA) following the manufacturer's instructions. Proteins were separated on 12% sodium dodecyl sulfate-polyacrylamide gels and transferred to polyvinylidene difluoride membranes (EMD Millipore, Darmstadt, Germany) The membranes were blocked and incubated with primary antibodies to GLUT2 (bs-0351r, Bioss, Woburn, Mass., USA) and N+-K+ ATPase (#3010S, Cell Signaling Technology, Danvers, Mass., USA). After three washes in PBST, membranes were incubated with HRP-conjugated secondary antibodies (Enzo Life Sciences, Farmingdale, N.Y., USA) for 1 h at room temperature. Protein bands were detected using ECL reagent (Thermo Scientific) and then visualized on films.

### Statistical Analyses

Data are presented as mean±standard deviation. The statistical significance of differences between means was examined by one-way analysis of variance (ANOVA) followed by Tukey's test. P<0.05 was considered statistically significant. ***P<0.001 compared to the control group, ##P<0.005, #IIIIP<0.001 compared to the high glucose (HG) group.

1. A method for treating a subject suffering from or susceptible to diabetes, comprising:
administering to the subject an effective amount of a compound of Formula 1: 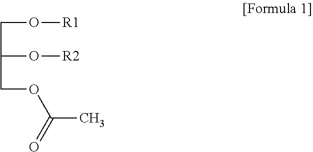
wherein R1 and R2 are independently a fatty acid residue of 14 to 22 carbon atoms.

2. The composition of claim 1 wherein R1 and R2 are independently selected from the group consisting of palmitoyl, oleoyl, linoleoyl, linolenoyl, stearoyl, myristoyl, and arachidonoyl.

3. The composition of claim 1 wherein the compound of Formula 1 is a compound of the following Formula 2:

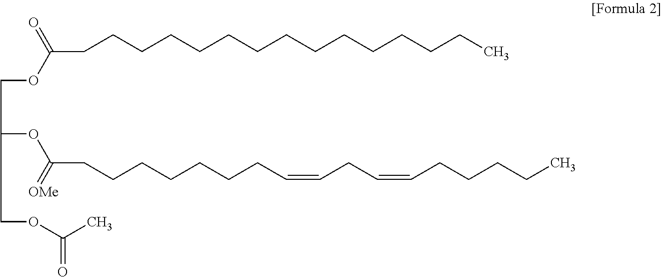

4. A method of treating a subject suffering from or susceptible to type 2 diabetes mellitus; diabetic dyslipidemia; impaired or inadequate glucose tolerance (IGT); impaired fasting plasma glucose (IFG); metabolic acidosis; ketosis; obesity; diabetic neuropathy; diabetic retinopathy and kidney disease; hyperlipidemia; arteriosclerosis; hypertension; insulin resistance; hyperglycemia; hypercholesterolemia; dyslipidemia, or syndrome X, the method comprising: administering to the subject an effective amount of a compound of Formula 1: 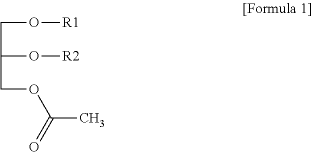
wherein R1 and R2 are independently a fatty acid residue of 14 to 22 carbon atoms.

5. The method of claim 4 wherein R1 and R2 are independently selected from the group consisting of palmitoyl, oleoyl, linoleoyl, linolenoyl, stearoyl, myristoyl, and arachidonoyl.

6. The method of claim 4 wherein the compound of Formula 1 is a compound of the following Formula 2:

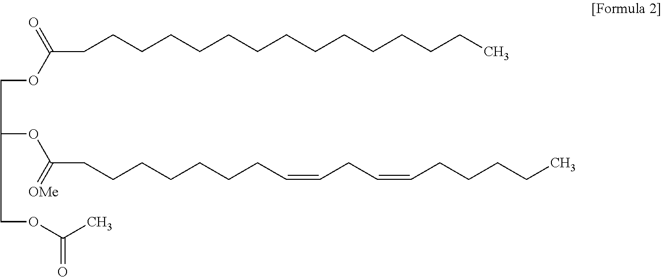

7. The method of claim 4 wherein:
the subject is identified as suffering from type 2 diabetes mellitus; diabetic dyslipidemia;
impaired or inadequate glucose tolerance (IGT); impaired fasting plasma glucose (IFG); metabolic acidosis; ketosis; obesity; diabetic neuropathy; diabetic retinopathy and kidney disease; hyperlipidemia; arteriosclerosis; hypertension; insulin resistance; hyperglycemia; hypercholesterolemia; dyslipidemia, or syndrome X; and
the compound of Formula 1 is administered to the identified subject.

8. The method of claim 1 wherein the subject is a human.

9. A kit comprising:
(a) a compound of Formula 1: 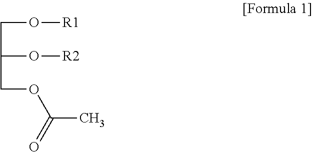
wherein R1 and R2 are independently a fatty acid residue of 14 to 22 carbon atoms; and
(b) instructions for using the compound for treating or preventing diabetes in a subject.

10. The composition of claim 9 wherein R1 and R2 are independently selected from the group consisting of palmitoyl, oleoyl, linoleoyl, linolenoyl, stearoyl, myristoyl, and arachidonoyl.

11. A kit of claim 9 wherein the compound of Formula 1 is (a)1 palmitoyl-2-linoleoyl-3-acetylglycerol (PLAG).

12. A kit of claim 11 wherein the kit comprises a therapeutically effective amount of PLAG.

13. A kit of claim 11 wherein the kit comprises written instructions for use of the PLAG.

14. A kit of claim 11 wherein the instructions are a product label.

15-20. (canceled)

21. A kit comprising:
(a)1-palmitoyl-2-linoleoyl-3-acetyl-race-glycerol (PLAG);
(b) one or more diabetic medications or diabetic treating agents that are distinct from PLAG; and
(c) instructions for using the PLAG for treating or preventing diabetes.

22-23. (canceled)

24. The method of claim 1 wherein the one or more compounds of Formula 1 are administered in combination with the one or more diabetic medications or diabetic treating agents that are distinct from the one or more compounds of Formula 1.

25. The method of claim 4 wherein the one or more compounds of Formula 1 are administered in combination with the one or more diabetic medications or diabetic treating agents that are distinct from the one or more compounds of Formula 1.

26. The method of claim 7 wherein the one or more compounds of Formula 1 are administered in combination with the one or more diabetic medications or diabetic treating agents that are distinct from the one or more compounds of Formula 1.
